(19) United States
(12) Patent Application Publication
(10) Pub. No.: US 2023/0414089 A1
(43) Pub. Date: Dec. 28, 2023

(54) DEVICES AND EXPERT SYSTEMS FOR INTUBATION AND BRONCHOSCOPY

(71) Applicant: REGENTS OF THE UNIVERSITY OF MINNESOTA, Minneapolis, MN (US)

(72) Inventors: Litty JOHN, Minneapolis, MN (US); Tjorvi PERRY, Minneapolis, MN (US); Abhimanyu KUMAR, Fremont, CA (US)

(73) Assignee: REGENTS OF THE UNIVERSITY OF MINNESOTA, Minneapolis, MN (US)

(21) Appl. No.: 18/035,918

(22) PCT Filed: Nov. 8, 2021
(86) PCT No.: PCT/US2021/072292

§ 371 Date: May 8, 2023

Related U.S. Application Data
(60) Provisional application No. 63/198,717, filed on Nov. 6, 2020.

(51) Int. Cl. A61B 1/267; A61B 1/008; A61B 1/00; A61B 1/06; A61B 1/05; A61B 1/015; A61B 1/07

(52) U.S. Cl. CPC A61B 1/2676; A61B 1/008; A61B 1/00097; A61B 1/0676; A61M 16/0488; A61B 1/00135; A61B 1/015; A61B 1/07; A61B 1/05

(57) ABSTRACT

Devices and Expert Systems for Intubation and Bronchoscopy incorporating a robotic scope and a self-learning neural network via a processing chip in the handle of the device. Sensors in a distal portion of the robotic scope provide input to the processing chip that determines future policies according to the current data received and past data stored. The distal portion of the device is directed to a target by a robotic scope directed by the processing chip.

### TECHNICAL FIELD

The present disclosure relates generally to devices for mapping or acting upon enclosed passageways, which can navigate using both optical and non-optical systems. Embodiments incorporate machine learning to better navigate such passageways. In some embodiments, intubation system is provided for insertion into a patient's trachea, and more specifically a self-learning intubation device that can efficiently navigate through an esophagus or trachea.

### BACKGROUND

Endotracheal intubation in the operating room, the intensive care unit or in the field is a challenging and potentially traumatic procedure that requires significant expertise. Recent technologic advances safely and effectively placing endotracheal tubes include video laryngoscopy and fiber optic bronchoscopes. However, these technologies continue to require significant user expertise and experience and have the potential to cause airway trauma with adverse hemodynamic consequences.

Currently available devices, including laryngoscopes, video laryngoscopes (VL) and fiber optic bronchoscopes (FOB), provide a 2-dimensional magnified, real time viewing of anatomic airway structures at zero-degrees. To safely and correctly place the endotracheal tube, the operator is required to identify key anatomic structures. In situations when key structures are not readily visible, the operator is at a significant disadvantage and often unable to place the endotracheal tube. Blind digital intubations were performed in the past and a number of blind insertion airway devices (BIAD), such as a laryngeal mask airway, a laryngeal tube, or a Combi-tube, are available for emergencies, but have not replaced conventional direct laryngoscopy (DL), VL or fiber optic bronchoscopes (FOB). Moreover, current technology is unable to capture images beyond 80-100 degrees of angle of viewing at the tip, blinding the user to relative airway anatomy. Current technology also lacks perception of depth.

Due to the difficulty of placement and the poor maneuverability of the tube, current devices (e.g., laryngoscopes) cause trauma to airway structures with potential for bleeding that can further jeopardize a difficult airway. The devices and systems of the present disclosure may therefore provide for reduced airway trauma. Current devices also can require significant pressure on airway structures during intubation that causes unfavorable hemodynamic response (e.g., tachycardia and hypertension) that is harmful in patients, especially those with cardiac disease.

### SUMMARY

Thus, there exists a present need in the art for improved intubation mapping or other guidance. There is a further need in the art to provide for a means of accelerating operator improvement in placing endotracheal tubes. Unfavorable hemodynamic responses may be reduced by the smart intubation system of the present disclosure. The devices and systems of the present disclosure can enable endotracheal intubation even in the presence of fluid in the airway (secretions, blood, vomit), that can jeopardize the view when using existing technology, making endotracheal intubations challenging.

To address the significant gaps in the art described above, disclosed herein are “self-learning” endotracheal intubation (ETT) devices and systems incorporating artificial intelligence and machine learning to effectively and efficiently guide the user, regardless of experience, through the patient's trachea.

Incorporating depth and spatial perception along with improved vision by widening the field and capturing the relative anatomy, the devices and systems of the present disclosure enable an operator to intubate efficiently and with ease. Since improved vision alone cannot guarantee efficient intubation, passage of the ETT may be difficult despite improved vision or spatial perception and needs to be integrated with greater degrees of freedom of the intubating device for consistently successful endotracheal intubations. Better maneuverability also reduces unfavorable hemodynamics such as tachycardia and hypertension from excessive pressure applied on the airway structures associated with laryngoscopy, making it more tolerable in patients with cardiac disease. It can also reduce trauma that may further compromise the airway, increasing the need for intervention and incurring additional costs.

In embodiments, the device is user agnostic and can demonstrate greater precision with each encounter.

Existing devices used for endotracheal intubation require significant expertise and experience, and the advantages provides by the present disclosure are many-fold.

Devices and Expert Systems for Intubation and Bronchoscopy described herein captures vision of relative anatomies of the airway, whereas conventional technologies are unable to capture images beyond 80-100 degrees of angle of viewing at the tip. Further, conventional technologies do not provide information about the relative anatomies of the airway. The devices and systems of the present disclosure incorporate spatial orientation and depth of perception, which is not possible with current devices that provide a magnified two-dimensional view.

There have been significant advances in machine learning that have led to intelligent devices across a broad spectrum of applications starting from home devices to commercial drilling tools. Specifically, robotics and autonomous agent techniques have made commercial and daily tools smarter at their tasks, eliminating many of the errors introduced by their human operators. Despite these recent advances there have been no applications of these techniques in the crucial field of medical devices, where human errors lead to much more devastating losses and, in the worst-case, loss of life itself. It makes it imperative to build medical tools that incorporate these new ideas to make the devices smarter and less error prone. The present disclosure describes an intubation system that navigates from the top of the mouth or the nose to a patient trachea with a low or least amount of necessary human guidance. The intubation system disclosed herein relies solely on an autonomous agent to guide the device through the airway with a low or least amount of human intervention. In some embodiments, the intubation device “crawls” through the airway, bypassing any natural as well as unnatural obstructions such as epiglottis, blood clots, pharyngeal or laryngeal mass, vomitus, etc.

In embodiments, the intubation system comprises a robotic scope or cable that is flexible and with greater degrees of freedom along its distal length that can be routed through a patient's airway during intubation. Visual sensors and/or non-visual sensors are distributed along the distal part of the robotic scope to provide information on airway pattern as well as give real time vitals of the patient such as blood oxygen saturation (SPO2), end tidal carbon dioxide (ETCO2) and pulse rate. The data from the sensors is transmitted to a processing unit attached to the device that applies a set of algorithms to identify a preferred, optimal, or correct pathway to the trachea. As the robotic scope navigates through the airway, it will display on the user interface the name of the structures it has identified along with the probability of correct identification based on prior airway patterns. Based on these outputs, the intubation device can either steer the endotracheal tube into the trachea or direct the user to manually guide the endotracheal tube into the trachea. In addition to knowledge from prior airway patterns, embodiments of the intubation device can use reinforced learning and become more intelligent with subsequent encounters.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1a depicts a robotic scope, according to an embodiment.

FIG. 1B is an example of the tip of the robotic scope shown in FIG. 1a.

FIG. 2 is an associated autonomous software agent for the smart intubation system, according to embodiments of the present disclosure.

FIG. 3 is a flowchart corresponding to devices and expert systems for intubation and Bronchoscopy, according to embodiments of the present disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The present disclosure relates to a smart intubation device and system to intubate a patient guided by outputs from sensors and, further, that facilitates machine learning with each airway encounter. The inputs from the sensors can translate to actual course of movement by the device.

Outside hospitals, this device may be used by paramedics in the field or aboard an ambulance and in military settings, for example. Outside endotracheal intubation, this device can be used for bronchoscopy, placement of nasal or orogastric tubes, vocal cord studies for ENT surgeons, and Foley catheter placement.

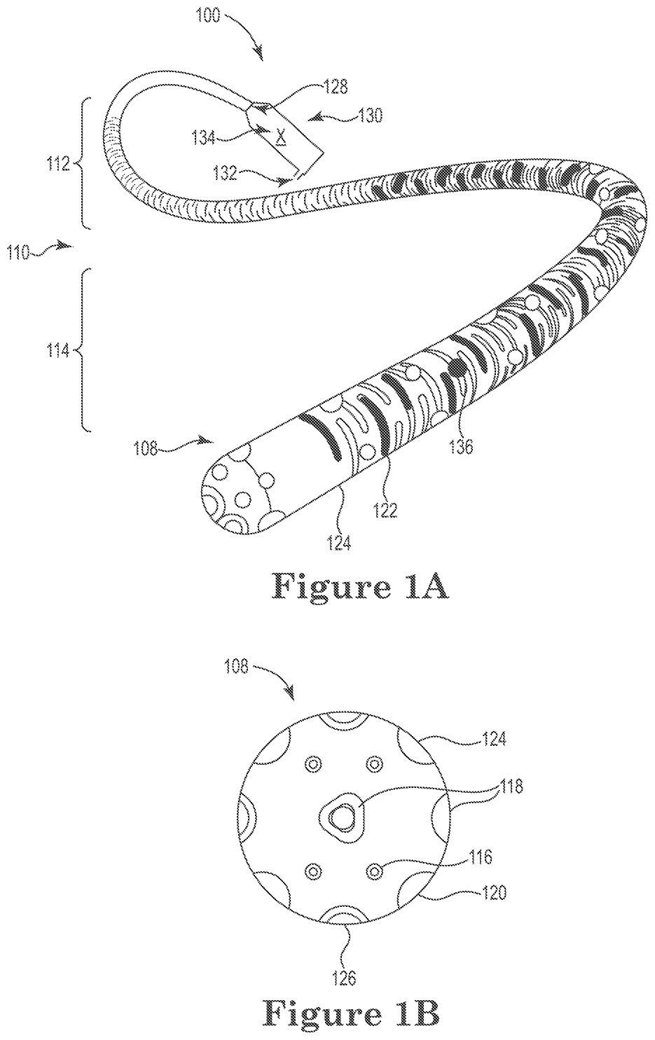

FIGS. 1a and 1b show robotic scope 100, which is configured to provide data on an intubation process to a user interface, a controlling unit, a processing unit, and a supporting body frame. Each of these components is described in more detail below.

Robotic Scope

Robotic scope 100 includes a tip 108, a body 110, a sleeve cover 124, a handle 130, a chip 128, and an oxygenation and suction system 126 (port at the distal end) and 132 (connector to the source at the proximal end). Throughout this disclosure, the term “scope” is used in the manner it would be understood by a medical practitioner such as a pulmonologist. Bronchoscopes or endoscopes use a form of cable that allows examination or intervention of hollow structures such as airway or gastrointestinal tract, which can also be incorporated into the systems and devices described herein.

The tip 108 is non-traumatic achieved by a J-tip, coil like a spring, or a balloon. This model has been adopted in the past to be used for various purposes within the body (J-tip for guidewire for central venous catheter insertion, coiled wires for cardiac pacing leads, balloon tip for Swan-Ganz catheter) to reduce the risk of perforation. The tip 108 can be any one of (or a combination of) a full spring coil tip, a polymer jacket over spring coil, a full polymer tip, and a micro-cut nitinol sleeve.

The body 110 incorporates robot wired mechanics to control the navigation via the IOT chip 128 output signal. The body 110 can be further divided into a proximal part 112 and a distal part 114. The proximal part 112 is defined herein as the part of the body 110 that would be arranged closer to the user/operator during an intubation procedure. The distal part 114 is defined herein as the part of the body 110 that would be arranged closer to the target or patient during an intubation procedure. The distal part 114 can have one or more light sources 116, such as LED, incandescent, or halogen light sources or a combination thereof. The distal part 114 can also one or more cameras 118. In embodiments with multiple cameras 118, the robotic scope 100 may implement stereoscopic vision. As used herein, the term “camera” can refer to any number of types of visual sensors that will be understood by a person having ordinary skill in the art to be usable in bronchoscopy or intubation procedures.

The visual sensors or cameras 118 can include at least five high-definition micro cameras in one embodiment, to cover the four possible sideway movements (i.e., up, down, left, right) and a camera 118 at the front to look forward. Each of these five cameras can be paired with a corresponding micro-LIDAR sensor for mapping the internal surface of the upper airway. The four side sensor pairs can be located at a distance of less than an inch form the distal end 108 in one embodiment, along the circular periphery and equidistant from one another. The fifth sensor pair can be installed at the distal end (i.e., the distal most part of tip 108) encompassed with a compass to give orientation of the camera within the airway. In this way, a user can visualize a broad field of airway. This also verifies endotracheal intubation by confirming pertinent visual positives (trachea) and negatives (esophagus).

Non-optical sensors 120 can also be included and can include sensors configured to detect ultrasound waves, electromagnetic waves, or other non-visual signals. The output of these sensors 120 can be reconstructed to produce 3D images, either alone or in combination with the visual signal detected by the visual sensors or cameras 118. These sensors can also include mechanical sensors such as pressure sensors or lateral airflow sensors.

Application of these non-optical sensors 120 include navigating through airway in the absence of a good visual output in the presence of secretions/blood/vomitus, identifying movements of the chest during ventilation (to confirm endotracheal intubation), and identifying the correct site for surgical airway (e.g., precise location of cricothyroid membrane) when the device is placed on the neck or oropharynx. A subset 136 of the non-optical sensors 120 are arranged along the side-wall of the device to measure body vitals, in embodiments. The non-optical sensors 120 can also include the mechanical sensors such as pressure sensors or lateral airflow sensors mentioned above. By detecting airflow and pressure on the sides of the robotic scope 100, the device can determine which portions are against an airway wall or other structure, and which sides are adjacent the region of airflow. This provides advantages to the operator (or a valuable software input) because the robotic scope 100 can be navigated to keep the airflow path open, or to provide indications of blockages. Furthermore, some procedures may involve muscle relaxant or sedation such that include a risk that the patient will stop breathing altogether, which can also be detected by such sensors.

Real time mainstream capnography (monitoring of carbon dioxide levels in the airway during anesthesia) with continuous quantitative waveform along with patient's oxygen saturation and pulse rate can be measured by sensors within the body and displayed on a screen (not shown). It should be understood that any of a variety of screens could be used, either those that are directly electrically connected to the robotic scope 100 or those that are remote from it (such as a wirelessly connected TV screen, computer monitor, or instrument panel, among others).

Multiple joints 122 one millimeter or its fractions apart are shown in FIG. 1a that enable articulation along the distal part 114. These articulation joints 122 enable 6-10 degrees of freedom and complete control of movement at the tip 108 as well as along the distal part 114 of the robotic scope 100. The robotic scope 100 can therefore navigate through both open and collapsed structures either by crawling (contact point is floor) or hanging down (contact point is roof), or any other placement within an airway. It should be understood that the spacing of these articulations, as well as the amount of angular change at each one, can be varied within machining tolerances to any desired level.

Conventional bronchoscopes generally limit degrees of freedom to no more than two to allow the “push” pressure applied by a user to drive the scope toward the target despite the natural resistance of a patient's body to such an invasion. Devices contemplated herein are not bound by this limitation of conventional systems, as they can “crawl” toward a target through the direction of the IOT chip 128. By “crawl” or “crawling,” as used throughout this application, it is meant that the robotic scope 100 can move under a “pull” force instead of the conventional mechanism based on “push” force, thereby allowing for greater degrees of freedom to circumvent obstacles or bodily resistance, rather than driving past it. With the help of the sensors added at the distal end the AI algorithm will provide in total ten degrees of freedom: 1) move up, 2) move down, 3) move right, 4) move left, 5) move at 45 degrees between up and left, 6) move at 45 degrees between down and left, 7) move at 45 degrees between up and right, 8) move at 45 degrees between down and right, 9) move forward, and 10) backward.

Sleeve cover 124 can be made of a tissue-compatible material that is adherent to the body of the robotic scope 100 and covers the tip 108. Sleeve cover 124 can be used to smooth the passage of robotic scope 100 through an airway and reduce trauma.

Handle 130 is either a non-detachable component or a detachable component that can be reattached to the proximal part 112. The detachable component allows the user to detach the handle from the robotic scope to thread in the endotracheal tube over it once its tip is in place. The detachable handle 130 as shown presents an improvement over conventional systems, in which the endotracheal tube must be preloaded on the scope prior to its insertion into the airway. Alternately, in disposable bronchoscopes, a common approach is to simply cut the proximal end of the scope off the handle once its tip is in position to allow threading in the endotracheal tube. A wireless version is also contemplated that does not include a handle.

Chip 128 can be any of a variety of processors, including those with telemetry functionality such as an Internet of Things (IOT) compatible chip. An IOT computing chip 128 is incorporated within the robotic scope at the proximal end of the body or within its handle. The autonomous agent rims as software on this chip 128. The chip 128 is designed to compute fast possible action sets for navigation based on the agents deep neural network trained on separate set of GPUs. The chip 128 takes input from visual sensors 118 and non-visual sensors 120 installed at the distal part 114 of the robotic scope. The chip 128 provides control and flexibility to the intubation device without losing its degrees of freedom. The chip 128 enables more flexibility as well as better maneuverability than the current state of intubation in fiber optic devices.

Optionally, in some embodiments, a separate channel can be included in robotic scope 100 for oxygen delivery through passive ventilation and suction of fluid (126).

Referring now to a potential user interface, an optical image can be broadcasted by means of a screen or a projector, such as to a detachable wireless big screen, or to two screens opposing one another in an inverted “V.” In embodiments, the display can be on a foldable screen that flips open similar to a book that opens to have two sides, one facing the user, other side facing an operating room. All audience in the operating room can then watch the procedure in real time. Alternatively, a multi-fold embodiment is contemplated that is expandable. The benefit of this alternative is that it may be visible to a larger audience, and the screen takes less space during storage. The display may also be a wall projection in two dimensions, or a mid-air three-dimensional projection, or a semicircular screen enabling three-dimensional viewing.

In embodiments, robotic scope 100 can include functionality to start recording when switched on to provide for such display. Additionally or alternatively, robotic scope 100 can be designed to manually mark a point of interest on the screen as a reference point on demand. A user can therefore easily create alert flags to avoid future mistakes and to help the robotic scope 100 (and others sharing a learning system with it) to learn the wrongly identified structures and improve.

Turning now to the controlling unit, movements of the distal part 114 of the robotic scope 100 can be controlled by the user via a controlling unit such as a joystick that may be located on the handle 134 or remotely accessed on the user interface or a remote-control device (not shown). The movement along the roll axis of the robotic scope 100 can be controlled manually or autonomously, in embodiments. In the manual mode, the user advances or retracts the device for forward or for backward movement respectively. In autonomous mode, the robotic scope can crawl down to its target with little or no operator intervention.

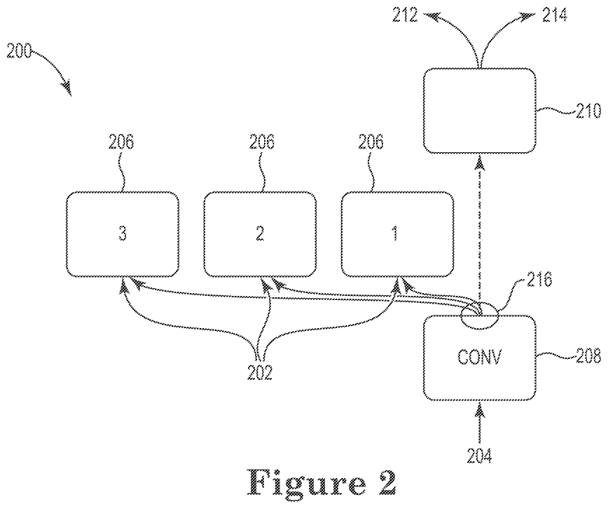

Turning now to the processing unit, this component can be located at the proximal part 112 of the robotic scope 100. The processing unit receives the input from the sensor pairs (118, 120) and performs inference on the trained model (as described below with respect to FIG. 2). The raw input enters the convolution layer 204 (FIG. 2) and the final action steps are relayed back to the tube 214 (FIG. 2) when the inference is done.

In some embodiments, the robotic scope 100 can be mounted on a supporting body frame. In order to facilitate transportation of the mounted device, various features or their combination can be incorporated: For example, the device can fly to user location like a drone or use different types of wheels to improve handling, speed and firmer ride, such as larger diameter wheels, tank treads or caterpillar tracks, large volume low pressure tires that include shock absorbent aspects, or low surface area contact three-dimensional wheels. Telescoping of the user interface enables the screen to be placed at an optimal vantage point to the user and the patient for easy visual access.

According to an embodiment, a method of operating this system for endotracheal intubation is insertion of the distal end of the device 114 directly into oral cavity. Once the tip 108 reaches an assigned target (passes beyond the vocal cord), the handle 130 is detached from the proximal end 112 and an endotracheal tube can be slid through the proximal end 112 towards the tip 108 using the Modified Seldinger technique. Another method of operating this device is by inserting the robotic scope 100 into a working channel of a fiberoptic bronchoscope (FOB) such that the tip of the robotic scope 108 projects outside the tip of the FOB. By this approach, the device will inherit most of the features of the robotic scope 100 such as broadening vision and increasing degrees of freedom. In this set up, the endotracheal tube has to be preloaded on to the FOB. The endotracheal tube can either be held proximally close to the FOB handle or the endotracheal can be held distally closer to the tip of the FOB stationed in the oral cavity until the tip of the robotic scope 108 reaches the target and the endotracheal tube slid over the FOB to its target position. Yet in another approach, the robotic scope 100 can be used like a stylet preloaded into an endotracheal tube. The robotic scope 100 can be locked in place at the proximal end of the endotracheal tube by means of an anchoring device so that the robotic scope 100 is held in place and does not slide up or down. The robotic scope-endotracheal tube assembly is advanced as an assembly. Upon reaching the target, the anchoring device is unlocked and the robotic scope 100 can be retracted back, keeping the endotracheal tube in position.

The current gaps in the art, including as described in the Background, have left a need in the field for a device with wider vision to capture relative anatomy and spatial perception of intubating device and finer control of intubating device to steer the endotracheal tube into the trachea.

Described above is an intubation device comprising a flexible robotic scope that can “crawl” through the airway. The part of the device inside the airway can comprise a flexible robotic scope that can be controlled at multiple points, providing degrees of freedom at multiple points and in multiple directions from the axis of the robotic scope. The distal end of the device has visual and/or non-visual sensor(s) that provide input about airway pattern. These inputs can be provided to an autonomous learning agent to resolve the deficiencies of conventional systems.

In embodiments, this intubation system may have surface-mapping tools such as cameras or sensors attached to it that provide the agent, which may be running on an IOT chip, with lossless compressed surface maps. The compression can ensure that the surface map input data is transferred quickly and its lossless-ness can ensure that the data retains its fidelity during compression. These surface maps data can be in the form of images or sensory data of the insides of the airways.

The devices described herein can be operated via an agent, such as a software-based agent, that takes input as surface maps in the form of images, sensor data, etc., and extracts representation of bodily entities, obstructions and pathways out of it. This aspect of the agent is referred to herein as the vision block. These entities in turn help guide the agent in finding policies to navigate the pathways and reach the trachea using reinforcement learning techniques. This aspect of the agent is referred to herein as a navigation-policy block. The vision block of the agent takes surface maps as input and extracts entities out of it. These entities include teeth, tongue, tonsils and the tonsillar pillars, epiglottis, arytenoids, aryepiglottic fold, etc. The policy block uses these entities to derive a feasible set of navigation policies. The policy block of the agent takes entities extracted at the current point of time, past entities seen, and past pathways taken to derive a set of possible actions to take. These actions can get more surface map data, turn left, turn right, go back, go forward, turn degrees between up and left etc. The ultimate aim of this block is to make the intubation device reach trachea. This decision to make turns is relayed from the IOT chip to distal end of the device via high tensile and high strength metal wires that act as pulley-lever mechanism. The abstract actions to make turns (left, right, up, down etc.) is translated into pulley-lever actions of varying degree of pull pressures on the connecting wires inside the software running on the IOT chip.

The agent can initially be trained on multiple sets of airway trachea navigation, which enables the agent to build a robust repertoire of possible navigation policies. This robust policy set can help the agent navigate smoothly across various airway paths. The device can be initially trained in a set of airway situations, and it can subsequently self-train to navigate unknown and complicated airways efficiently. Reinforced learning may enable the device to navigate airways with unanticipated complications such as small dimensions caused by edema, stenosis or obstructions, or false passages.

The agent can be enabled to transfer learning, such as by using prior navigation experience from a set of airways as a reference, to be able to navigate successfully through a new environment or unknown set of airways.

Software running on chip 128, such as an autonomous software agent for positioning distal end 108 or other software (e.g., imaging software) depending on the desired characteristics or functionality of intubation device 100, can be optimized to run on the tiny IOT chip 128, thus consuming little power compared to normal computing CPU chips. The software agent may first be trained on a set of GPUs on past airways navigation data. This trained software may then be installed to operate on the IOT, or other computing or processing, chip 128 to perform computations in real time, for example to determine or develop a next set of policy and actions for navigation. The software can be made robust such that it can run both on a server of GPUs in training time and a tiny IOT chip in test time, e.g., in the course of performing an intubation. This ability to run on different sets of computing devices (e.g., GPUs and IOT/Micro-chips) greatly enhances the ability to assemble and mass produce such devices because training an autonomous agent on non-GPU processing units is less efficient and slower.

Referring now to FIG. 2, an example autonomous agent 200 is shown, according to embodiments of the present disclosure. In embodiments, agent 200 may receive multiple kinds of inputs. In the example agent 200, two possible inputs are shown: past airways data 202 that can provide recorded information from past airways navigation surface maps and input surface maps 204 that can provide data of surface maps at the current timestamp during the navigation of an airway.

Agent 200 can use input data, such as past airways data 202 and current surface map data 204, to inform one or more sets of neural network parameters 206. Neural network parameters 206 can be trained on previously seen airways, such as past airways data 202. The input surface maps 204 may pass through a convolutional neural network block 208 to extract entities 216 from these current maps 204. A policy and action evaluation block 210 can receive, as input, neural network parameters 206 corresponding to policies taken in past airway navigations 202 as well as the current entities 216 extracted from surface maps 204 and combines them to compute next set of policies 212 and actions 214 to take.

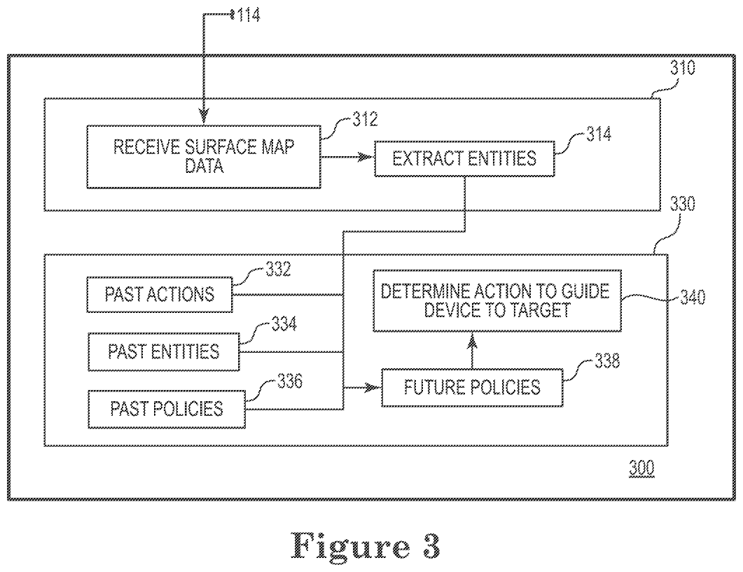

Referring now to FIG. 3, a block diagram of an example autonomous agent 300 is shown, according to embodiments of the present disclosure.

Autonomous agent 300 may comprise at least two major blocks, a vision block 310 and a policy block 330. The vision block 310 can take surface map data 312 obtained via cameras and sensors 114 and extracts entities 314 or obstructions relevant for navigation. These entities 314 can be natural body parts such as wall of the mouth, epiglottis, tongue etc. or unnatural obstructions such as vomitus, contusion, blood clots etc. A combination of networks, such as multi-layered deep or convolutional neural networks, can be used to extract these entities 314. Extracted entities 314 can be passed on to policy block 330. Policy block 330 can guide the device to a target destination, e.g., the trachea, using received entities 314. Policy block 330 can use inputs including, but not limited to, past actions taken 332, past entities seen 334, entities 314 obtained at the current timestamp, and past policies 336 to obtain the future set of policies 338 for navigation. Future policies 338 can in turn determine an action 340 to be taken such as go left, turn right, decrease speed, turn back, move forward etc. Policy block 330 can use deep reinforcement learning, for example, with a multilayered neural network to obtain policies and actions. Deep neural networks, which may be used for policy block 330 as well as vision block 310, can be trained on past datasets, such as past actions 332 or past entities 334, for airways navigation and apply one or more learned patterns 336 combined with present learning 314, e.g. data obtained in the course of the current navigation, to guide 340 the intubation device to its destination.

The autonomous agent described can learn a policy set on past trachea airway navigation. Each data point in a given dataset may represent one instance of an intubation device being guided through the airways to the trachea. A vision block can also be trained on a labeled set of data for different possible entities encountered in the airways. While navigating, the autonomous agent can obtain images or sensor data from one or more cameras or sensors on the distal end of the intubation device. These images and sensor snapshots can be superimposed over each other to obtain a more robust surface map for improved navigation. This image and sensor data can be compressed before being sent from cameras/sensors to the chip to provide faster and improved quality transmission of the data points.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of U.S.C. § 112(f) are not to be invoked unless the specific terms “means for” or “step for” are recited in a claim.

1. A device for endotracheal intubation or diagnosis comprising:
a handle
a user interface;
a processor;
a robotic scope comprising a plurality of articulation joints and a plurality of sensors, and defining a proximal end and a distal end;
the distal end of the robotic scope having a tip that includes a subset of the plurality of sensors; and
wherein the plurality of sensors are in communication with the processor such that the processor relays information based upon data from the plurality of sensors to the user interface.

2. A system for endotracheal intubation using the device or system of claim 1, comprising:
an agent comprising a vision block and a policy block;
the vision block receiving data from the plurality of sensors and extracting representations of structures and pathways around the distal end of the device;
the policy block receiving the representations from the vision block deriving a set of actions to take.

3. The device or system of any of the above claims wherein the policy block further relies upon past data when deriving a set of action to take.

4. The device or system of any of the above claims wherein the processor comprises the vision block.

5. The device or system of any of the above claims wherein the processor comprises the policy block.

6. The device or system of any of the above claims wherein the processor comprises the agent.

7. The device or system of any of the above claims further comprising an IOT block.

8. The device or system of any of the above claims wherein the set of actions to take is relayed from the policy bock to the distal end.

9. The device or system of any of the above claims wherein the set of actions to take is relayed from the policy to the distal end via the IOT block.

10. The device or system of any of the above claims wherein the policy block performs interference on a trained model.

11. The device or system of any of the above claims wherein the processor performs interference on a trained model.

12. The device or system of any of the above claims wherein the policy block or the processor enters the set of actions to take into a convolution layer before relaying the set of actions to take to the distal end.

13. The device or system of any of the above claims wherein the distal end further comprises an operation system associated with each of the plurality of articulated joints.

14. The device or system of any of the above claims wherein the operation system is a pulley and/or a lever system.

15. The device or system of any of the above claims wherein the robotic scope is mounted on a supporting body frame.

16. The device or system of any of the above claims wherein the body frame further comprises one or more transport components.

17. The device or system of any of the above claims wherein the one or more transport components comprise one or more member of a group including drone flight and wheels.

18. The device or system of any of the above claims wherein the wheels comprise one or more members of a group including large diameter wheels, tank tread, caterpillar tracks, low volume-low pressure tires, wheels with shock absorbent aspects, and low surface area contact wheels.

19. The device or system of any of the above claims wherein the distal end of the robotic scope comprises a tip.

20. The device or system of claim 1 wherein the proximal end guides the distal end of the robotic scope in response to direction from the processor.

21. The device or system of any of the above claims wherein the proximal end guides the distal end of the robotic scope in response to direction from an operator.

22. The device or system of any of the above claims wherein the tip is an atraumatic tip.

23. The device or system of any of the above claims wherein the atraumatic tip comprises one or members of a group including a J-tip, a spring coil, and a balloon.

24. The device or system of any of the above claims wherein the spring coil comprises one or more members of a group including a full spring coil, a jacketed spring coil, a full polymer tip, and a micro-cut nitinol sleeve.

25. The device or system of any of the above claims further comprising a suction system and or an oxygenation system comprising a port at the distal end and a source connector at the proximal end.

26. The device or system of any of the above claims wherein the distal end comprises one or more light sources.

27. The device or system of any of the above claims wherein the one or more light sources comprise one or more members from a group including LED, incandescent, and halogen lights.

28. The device or system of any of the above claims wherein the distal end comprises one or more camera.

29. The device or system of any of the above claims wherein the robotic scope may implement stereoscope visions.

30. The device or system of any of the above claims wherein the one or more cameras comprises at least five high-definition (HD) cameras.

31. The device or system of any of the above claims wherein at least four cameras each covers one of four possible sideway movements of the device.

32. The device or system of any of the above claims wherein the at least four cameras are arranged as two sets of pairs.

33. The device or system of any of the above claims wherein the at least four cameras are less than one inch from the distal end.

34. The device or system of any of the above claims wherein the at least four cameras are around the circular periphery of the distal end.

35. The device or system of any of the above claims wherein the at least four cameras are equidistant from one another.

36. The device or system of any of the above claims wherein at least one camera is arranged on the distal most point of the tip.

37. The device or system of any of the above claims wherein the distal most point of the tip further comprises a compass.

38. The device or system of any of the above claims wherein one or more of the at least five HD cameras is paired with a corresponding micro-LIDAR unit.

39. The device or system of any of the above claims wherein the plurality of sensors comprises optical sensors and/or non-optical sensors.

40. The device or system of any of the above claims wherein the non-optical sensors comprise ultrasound and=/or electromagnetic sensors.

41. The device or system of any of the above claims wherein the processor reconstructs a 3D image of the device's surroundings based on data received from the non-optical sensors.

42. The device or system of any of the above claims wherein the reconstruction of the device's surroundings comprises a lossless compressed map.

43. The device or system of any of the above claims wherein the plurality of sensors comprise one or more sensors for measurement of body vitals.

44. The device or system of any of the above claims wherein the one or more sensors for measurement of body vitals provide real-time capnography.

45. The device or system of any of the above claims wherein the one or more sensors for measurement of body vitals provide a continuous quantitative waveform with patient's oxygen and/or pulse rate.

46. The device or system of any of the above claims wherein the plurality of articulated joints are one millimeter and/or its fraction apart.

47. The device or system of any of the above claims wherein the plurality of articulated joints enable two or more degrees of freedom.

48. The device or system of any of the above claims wherein the plurality of articulated joints enable at least six degrees of freedom.

49. The device or system of any of the above claims wherein the plurality of articulated joints enable at least ten degrees of freedom.

50. The device or system of any of the above claims further comprising a sleeve cover.

51. The device or system of any of the above claims wherein the sleeve cover is tissue compatible.

52. The device or system of any of the above claims wherein the sleeve cover is adherent to the robotic scope.

53. The device or system of any of the above claims wherein the sleeve cover covers the tip.

54. The device or system of any of the above claims wherein the handle is detachable.

55. The device or system of any of the above claims wherein the proximal end of the robotic scope attaches to the handle.

56. The device or system of any of the above claims wherein the robotic scope further comprises a separate channel for the delivery of oxygen through passive ventilation and suction.

57. A method of operating a system for endotracheal intubation using the device or system of any of the above claims, the method comprising:
inserting distal end of the device into oral cavity;
detaching a handle from the device in response to the tip reaching an assigned target; and
sliding an endotracheal tube through a proximal end toward the tip.

58. The method of claim 57, wherein sliding an endotracheal tube through a proximal end toward the tip comprises using a Modified Seldinger technique.

59. A method of operating the device or system of any of the above claims, the method comprising:
inserting the robotic scope into a working channel of a bronchoscope such that the tip of the robotic scope projects outside the tip of the bronchoscope; and
holding the device proximally close to the FOB handle or distally closer to the tip of the bronchoscope stationed in the oral cavity until the tip of the robotic scope reaches the target and the endotracheal tube slid over the bronchoscope to its target position.

60. The method of claim 59, wherein a fiberoptic bronchoscope

61. A method of operating the device or system of any of the above claims, the method comprising:
preloading the robotic scope into an endotracheal tube similar to a stylet;
locking the robotic scope in place at the proximal end of the endotracheal tube by means of an anchoring device so that the robotic scope is held in place and does not slide up or down;
advancing the robotic scope-endotracheal tube assembly as an assembly;
unlocking the anchoring device upon reaching the target; and
retracting the robotic scope keeping the endotracheal tube in position.
